(19) United States
(12) Patent Application Publication
(10) Pub. No.: US 2006/0083859 A1
(43) Pub. Date: Apr. 20, 2006

(54) Magnetic levitation system for coating a device, a method of using the system, and device made by the system

(76) Inventor: Todd Robida, Southbridge, MA (US)

Correspondence Address: KENYON & KENYON LLP, 1500 K STREET N.W., SUITE 700, WASHINGTON, DC, 20005, US

(21) Appl. No.: 10/969,449

(22) Filed: Oct. 20, 2004

(51) Int. Cl. B05D 3/02; B01J 19/08; B29C 71/04; A61F 2/82

(52) U.S. Cl. 427/372.2; 427/457; 427/532; 427/421.1; 118/300; 623/1.46

(57) ABSTRACT

A method of reducing coating defects is provided. A non-contact coating system is provided that includes a high-powered magnet adapted to suspend an object and an arrangement for creating a coating mist in a suspension zone of the magnet. A method is provided for holding a device during a coating process. The method includes applying a magnetic field to the device and contacting a coating material with the device. The magnetic field causes the device to be suspended in a coating zone. A medical appliance is provided having a coating applied by a method. The method includes positioning the medical appliance in a coating zone; suspending the medical appliance in the coating zone by applying a magnetic field; and contacting the medical appliance with the coating. An article of manufacture is provided that is produced by a method. The method includes moving the article into a suspension area; applying a magnetic field to hold the article in the suspension area; and creating a cloud of coating material in the suspension area. A method for applying a coating to an object is provided that includes suspending a mist of the coating in a magnetic field and dropping the object through the mist.

### FIELD OF THE INVENTION

The present invention relates to coating methods. More particularly, the present invention relates to a system and method for coating a device such as a stent using a magnetic levitation system.

### BACKGROUND INFORMATION

Medical devices may be coated so that the surfaces of such devices have desired properties or effects. For example, it may be useful to coat medical devices to provide for the localized delivery of therapeutic agents to target locations within the body, such as to treat localized disease (e.g., heart disease) or occluded body lumens. Localized drug delivery may avoid some of the problems of systemic drug administration, which may be accompanied by unwanted effects on parts of the body which are not to be treated. Additionally, treatment of the afflicted part of the body may require a high concentration of therapeutic agent that may not be achievable by systemic administration. Localized drug delivery may be achieved, for example, by coating balloon catheters, stents and the like with the therapeutic agent to be locally delivered. The coating on medical devices may provide for controlled release, which may include long-term or sustained release, of a bioactive material.

Aside from facilitating localized drug delivery, medical devices may be coated with materials to provide beneficial surface properties. For example, medical devices are often coated with radiopaque materials to allow for fluoroscopic visualization while placed in the body. It is also useful to coat certain devices to achieve enhanced biocompatibility and to improve surface properties such as lubriciousness.

Coatings have been applied to medical devices by processes such as dipping, spraying, vapor deposition, plasma polymerization, spin-coating and electrodeposition. Although these processes have been used to produce satisfactory coatings, they have numerous, associated potential drawbacks. For example, it may be difficult to achieve coatings of uniform thicknesses, both on individual parts and on batches of parts. Further, many conventional processes require multiple coating steps or stages for the application of a second coating material, or may require drying between coating steps or after the final coating step.

The spray-coating method has been used because of its excellent features, e.g., good efficiency and control over the amount or thickness of coating. However, conventional spray-coating methods, which may be implemented with a device such as an airbrush, have drawbacks. For example, when a medical device has a structure such that a portion of the device obstructs sprayed droplets from reaching another portion of the device, then the coating becomes uneven. Specifically, when a spray-coating is employed to coat a stent having a tube-like structure with openings, such as stents described in U.S. Pat. Nos. 4,655,771 and 4,954,126 to Wallsten, the coating on the inner wall of the tube-like structure may tend to be thinner than that applied to the outer wall of the tube-like structure. Hence, conventional spraying methods may tend to produce coated stents with coatings that are not uniform. Furthermore, conventional spraying methods are inefficient. In particular, generally only 5% of the coating solution that is sprayed to coat the medical device is actually deposited on the surface of the medical device. The majority of the sprayed coating solution may therefore be wasted.

In the spin-dipping process, a medical device is coupled to a spinning device, and then, while rotating about a central axis, the medical device is dipped into a coating solution to achieve the desired coating. This process also suffers from many inefficiencies including the unevenness of the coated layer and a lack of control over the coated layer's thickness.

In addition to the spray coating and spin-dipping methods, the electrostatic deposition method has been suggested for coating medical devices. For example, U.S. Pat. Nos. 5,824,049 and 6,096,070 to Ragheb et al. mention the use of electrostatic deposition to coat a medical device with a bioactive material. In the conventional electrodeposition or electrostatic spraying method, a surface of the medical device is electrically grounded and a gas may be used to atomize the coating solution into droplets. The droplets are then electrically charged using, for example, corona discharge, i.e., the atomized droplets are electrically charged by passing through a corona field. Since the droplets are charged, when they are applied to the surface of the medical device, they will be attracted to the surface since it is grounded.

One disadvantage of conventional electrostatic spraying is that it requires a complicated spraying apparatus. In addition, because conventional electrostatic systems use a gas to move the droplets from a source to a target, controlling the gas pressure is crucial for accurate coating. However, it is not easy to control the gas pressure so that the target surface is evenly and sufficiently coated without losing much of the coating solution.

Devices may be coated by a gas assisted spraying process. A polymer/drug combination may be dissolved in a solvent mixture. The solution may be sprayed onto the devices and a polymer/drug film may be formed when the solvents evaporate. The ability to apply thin coatings on products may be limited by the capabilities of a gas assisted spraying process. The coating may flow on the medical device prior to drying, thereby creating an uneven concentration of bioactive agent on the surface of the device. A gas assisted spraying process may have a high variability for thin coatings.

Conventional methods of coating stents or devices with a drug-polymer layer, such as spraying or dipping, may cause uneven and unpredictable wetting, and distribution and evaporation of the solvent molecules may result in a non-uniform coating. The drying of the coating may lead to cracking and/or points of stress in the coating. A non-uniform coating may lead to the unit failing Kinetic Drug Release (KDR), drug uniformity and coating thickness specifications.

Conventionally, stents are coated using a nozzle to apply a solution containing a polymer and drug. The stent is held as it is moved in front of the spray nozzle by a fixture comprised of fine wires which make contact with the stent struts. Coating defects can occur at the contact points with these fine wires of the fixture.

There is, therefore, a need for a cost-effective method of coating devices that results in thin, uniform, defect-free coatings and uniform drug doses per unit device. Each of the references cited herein is incorporated by reference herein for background information.

### SUMMARY

A method of reducing coating defects is provided. A non-contact coating system is provided that includes a high-powered magnet adapted to suspend an object and an arrangement for creating a coating mist in a suspension zone of the magnet.

The system may include an air mover adapted to at least one of insert the object into the suspension zone, move the object within the suspension zone, and remove the object from the suspension zone. The system may include a magnetic levitation arrangement adapted to at least one of insert the object into the suspension zone, move the object within the suspension zone, and remove the object from the suspension zone.

The system may include a robotic arm for at least one of placing the object in the suspension zone, dropping the object into the suspension zone, and retrieving the object from the suspension zone. The system may include a conveyor system for dropping the object into the suspension zone and/or recovering the object after the magnet is powered down. The system may include a vision system adapted to track the coating mist and/or the object; a processor electrically coupled to the vision system; and a database electrically coupled to the processor.

The system may include a weigh station adapted to weigh the object at least one of before and after the object is suspended in the suspension zone. The system may include an arrangement for drying the object, the arrangement for drying the object comprising at least one of an air flow arrangement, a heater, and a vacuum chamber.

The arrangement for creating the coating mist may include a low-pressure nozzle, an ultrasonic nozzle, an electrostatic arrangement for imparting an electrostatic field to a mist, a magnetic levitation mist movement arrangement, and/or an airflow mist movement arrangement. The high-powered magnet may be liquid cooled and/or donut-shaped.

A method is provided for holding a device during a coating process. The method includes applying a magnetic field to the device and contacting a coating material with the device. The magnetic field causes the device to be suspended in a coating zone.

The method may further include drying the coating material on the device. The drying may be achieved by waiting a predetermined time period, creating a vacuum around the device, heating the at least one device, and/or flowing a gas over the device. The magnetic field may be applied by a liquid cooled magnet. The method may further include moving the device into, out of, and/or within the coating zone by an airflow arrangement and/or a magnetic levitation movement arrangement.

The operation of contacting the coating material with the device may include spraying the coating material, transporting a mist of the coating material, and/or aerosolizing the coating material with an ultrasonic nozzle. The applying of the magnetic field to the device and the contacting of the coating material with the device may be performed simultaneously.

A medical appliance is provided having a coating applied by a method. The method includes positioning the medical appliance in a coating zone; suspending the medical appliance in the coating zone by applying a magnetic field; and contacting the medical appliance with the coating.

Contacting the medical appliance with the coating may further include spraying the coating with a low pressure spray nozzle, aerosolizing the coating with an ultrasonic nozzle, and/or providing an electrostatic charge to a mist of coating material.

An article of manufacture is provided that is produced by a method. The method includes moving the article into a suspension area; applying a magnetic field to hold the article in the suspension area; and creating a cloud of coating material in the suspension area.

The moving of the article into the suspension area may be assisted by a magnetic levitation arrangement and/or an air-flow arrangement. The method may further include moving the article out of the suspension area by a magnetic levitation arrangement and/or an air-flow arrangement.

The method may further include moving the article within the suspension area by a magnetic levitation arrangement and/or an air-flow arrangement. The method may further include moving the cloud of coating material in the suspension area by adjusting the magnetic field, creating a further magnetic field, and/or creating an air-flow in the suspension area.

A method for applying a coating to an object is provided that includes suspending a mist of the coating in a magnetic field and dropping the object through the mist.

### BRIEF DESCRIPTION OF THE DRAWINGS

FIG. 1 is a schematic representation of an exemplary embodiment of the present invention.

FIG. 2 is a schematic representation of an exemplary system for performing an exemplary method according to the present invention.

FIG. 3 is an alternative exemplary embodiment of the present invention.

FIG. 4 is a flowchart for performing an exemplary method of the present invention.

### DETAIL DESCRIPTION

Use of a non-contact coating and clamping method could minimize handling defects and provide a coating that is free of defects. A possible method to eliminate the contact point coating defects is to use a non-contact method of fixturing the stent during the coating operation. This could consist of a system by which the stent is held by a high-powered magnetic field in a coating chamber while the coating is applied.

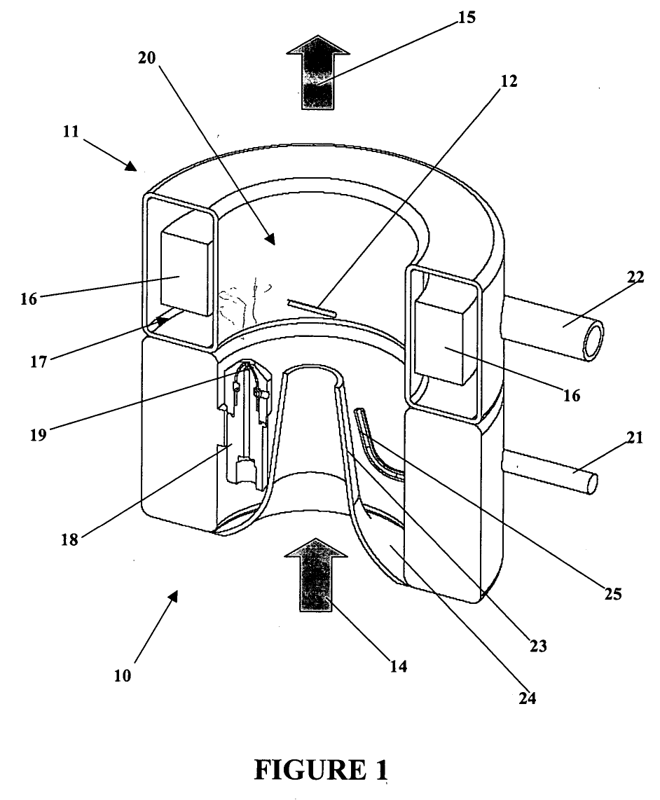

FIG. 1 is a schematic representation of an exemplary embodiment of the present invention. FIG. 1 shows mag lev coater 10, including magnet housing 11, being used to coat stent 12. Stent 12 is suspended in suspension zone 20 of mag lev coater 10. Magnet housing 11 houses magnet 16 and liquid chamber 17 which may accommodate liquid nitrogen or any other appropriate liquid coolant. Liquid chamber is supplied with liquid coolant by liquid source 22. Magnet 16 may be an open core, ring-type or donut-shaped magnet, to enable the creation of suspension zone 20 in a central area of magnet housing 11.

FIG. 1 also shows that mag lev coater 10 includes guidance nozzle 23 which may assist articles to be guided into suspension zone 20. For instance stent 12 may be injected into mag lev coater 10 in the direction of entrance arrow 14 by an air pressure mechanism and, when stent 12 enters suspension zone 20, the effect of magnet 16 may enable stent 12 to be held in a suspended state. The air pressure mechanism or a further air pressure mechanism may be activated to remove stent 12 from suspension zone 20 following coating and/or drying. Stent 12 may be removed in the direction of exit arrow 15. Alternatively, stent 12 may be introduced to mag lev coater 10 from above in the direction opposite to exit arrow 15, and/or may be removed from mag lev coater 10 in the direction opposite to entrance arrow 14.

Nozzle system 18 of FIG. 1 may provide a coating material into mag lev coater 10 via low-pressure nozzle 19. Alternatively, low-pressure nozzle 19 may be an ultrasonic nozzle or any other type of appropriate nozzle. Low-pressure nozzle 19 may impart an electrostatic charge to the coating material before ejecting it to promote adherence to stent 12 and/or to inhibit agglomeration in suspension zone 20. Additionally or alternatively, low-pressure nozzle 19 may eject coating material that includes magnetic material and/or microspheres to prevent agglomeration of the coating material. Nozzle system 18 is shown positioned in a low position directed upwards. Alternatively, nozzle system 18 may be positioned above mag lev coater 10 and directed downward, or in any other appropriate orientation. Nozzle system 18 may be supplied with coating and/or pressurized gas by nozzle fitting 21.

Guidance nozzle 23 may be coupled to coating recovery unit 24, which may serve to recover coating that settles out of suspension zone 20 over time or due to a powering down of magnet 16. Coating recovery unit 24 may include a drain and/or a mechanism for recycling and/or reusing any coating that is recovered. Positioning gas jet 25 may be used to position stent 12 in suspension zone 20. Positioning gas jet may use small bursts of air, nitrogen, or any other appropriate gas to move and/or rotate stent 12 in suspension zone 20. Positioning gas jet 25 may also be used to move coating particles that are suspended in suspension zone 20. Additionally or alternatively, magnet 16, or another supplementary magnet, may be used to move stent 12 and/or a coating mist positioned in suspension zone 20. Magnet 16 may be pulsed out of phase in order to impart an angular velocity to stent 12 to increase the amount of coating material that contacts a surface of stent 12 and to thereby improve the coating on stent 12. Additionally or alternatively, positioning gas jet 12 may provide heated air to suspension zone 20 to increase the drying rate for a coating on a surface of stent 12. Additionally or alternatively, a radiant heat and/or inductive heating arrangement may be positioned inside, above, or below mag lev coater 10 to increase the drying rate for the coating on the surface of stent 12.

The stent may be moved through a dense mist of coating solution that is also suspended in the magnetic field. Alternatively, a low-pressure (less than 3 PSI) spray nozzle may be used to minimize disturbance of the position of the stent in the magnetic field. The stent may be moved or rotated slightly using positioning jets as required. Other parts of the system could consist of air assisted transport of the stent to the pre- and post-weigh stations. Air assisted transport help to minimize handling defects.

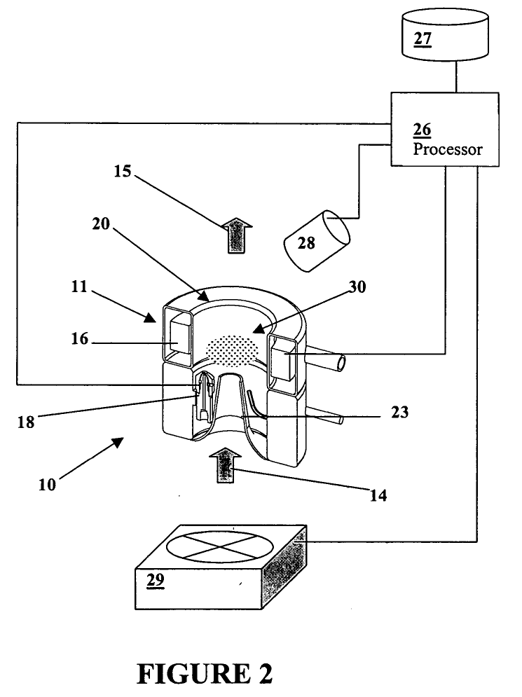

FIG. 2 is a schematic representation of an exemplary system for performing an exemplary method according to the present invention. FIG. 2 shows mag lev coater 10 including magnet housing 11 positioned above air mover 29. Air mover 29 may move a stent, another medical device, and/or articles, individually or in groups, in the direction of entrance arrow 14 up into mag lev coater 10, guided by guidance nozzle 23. Nozzle system 18 may be positioned in mag lev coater 10 to provide mist 30 of coating material in suspension zone 20. Magnet 16 in magnet housing 11 may operate to magnetically levitate mist 30, a stent, and/or another article in suspension zone 20. Vision system 28, which may be an optical, infrared, or any other appropriate vision system, may monitor the position of mist 30, a stent, and/or another article in suspension zone 20. Processor 26 may be electrically coupled to vision system 28 and may control magnet 16 to maintain mist 30 and/or a stent or other article in suspension zone 20. Processor 26 may also control air mover 29 to move the stent or other article into suspension zone 20, and/or to remove the stent or other article from mag lev coater 10 in a direction of exit arrow 15. Processor 26 may also control nozzle system 18 to maintain an appropriate density for mist 30 in suspension zone 20 in response to a signal from vision system 29. Processor 26 may also be electrically coupled to memory 27, which may include a database of instruction and/or parameters for operating magnet 16, air mover 29, nozzle system 18, and/or any other appropriate system. Processor 26 may operate any of the systems within its control in response to data received from vision system 28 and/or any other appropriate sensor system.

Stents, other medical devices, and/or articles, individually or in groups, may be brought in from the bottom of the spray chamber by air assist or dropped in from above via a robot. The stent may be held in suspension in a high powered magnetic field. The coating solution may be sprayed into the chamber by one or more nozzles continuously or injected once before the stent is loaded. The mist consisting of the coating solution may be held in the magnetic field with the stent. The stent may move in the spray or suspended mist from the movement of spray gas or via a set of positioning jets.

Alternatively, stents may be introduced into mag lev coater 10 from the bottom by air mover 29, suspended while being coated by magnet 16, then dropped out of mag lev coater 10 in a direction opposite to entrance arrow 14 simply by powering down magnet 16. In other alternatives, mag lev coater 10 may be operated to maintain mist 30 in suspension area 20 and a conveyor belt or robotic arm may drop a stent or other articles from the top in a direction opposite to exit arrow 15. The stent or other article may pass through mag lev coater 10 and through mist 30 and may be coated thereby. Magnet 16 may in this situation be operated at a power that is insufficient to maintain the stent or other article in suspension zone 20 but is powerful enough to maintain mist 30 in suspension zone 20. After passing through mist 30, the stent or other article may pass out through guidance nozzle 23 (which may, alternatively, be reversed in orientation) and out of mag lev coater 10 in a direction opposite to entrance arrow 14. The stent or other article may thereafter be stopped by an air cushion, another magnet, or by any other appropriate method.

Coating parameters that may be adjusted may include the volume of solution injected into the chamber, the chamber pressure and temperature, the length of time in the chamber, the mist density and the coating solution parameters such as percentage of solids, viscosity, mist droplet size, etc.

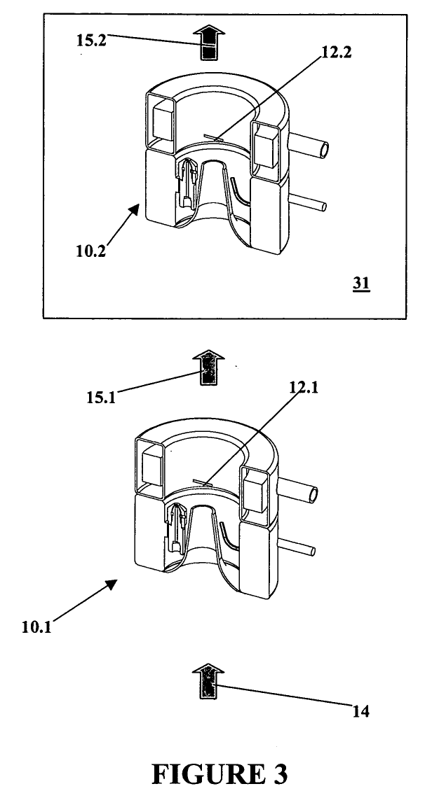

FIG. 3 is an alternative exemplary embodiment of the present invention that shows two mag lev coaters 10.1, 10.2 installed in series. FIG. 3 shows stent 12.1 suspended in mag lev coater 10.1 by the application of a strong magnetic field. Because of the strength of the magnetic field, stent 12.1 (or, alternatively, another article to be coated) is not necessarily composed of a magnetic material, or may be composed of only a weakly magnetic material. Stent 12.1 may have entered mag lev coater 10.1 from below in the direction of entrance arrow 14 at the impulse of another magnet, an air assist system, or any other appropriate system. After coating stent 12.1 in mag lev coater 10.1, stent 12.1 may be moved out of mag lev coater 10.1 in the direction of exit arrow 15.1 by another magnet, an air assist system, or any other appropriate system. Stent 12.2 is positioned in mag lev coater 10.2, which may arranged in the direction of exit arrow 15.1 from mag lev coater 10.1. Mag lev coater 10.2 may be arranged in vacuum chamber 31, which may provide a vacuum or partial vacuum to assist in the drying of the coating on stent 12.2. Alternatively, vacuum chamber 31 may be a radiant heat chamber, inductive heat chamber, or any other system to assist the drying of the coating on stent 12.2. After drying stent 12.2 or waiting an appropriate period of time, stent 12.2 may be removed from mag lev coater 10.2 by another magnet, an air assist system, or any other appropriate system in the direction of exit arrow 15.2. Alternatively, mag lev coater 10.2 may be used to apply a second coating to stent 12.2. The second coating may have the same or a different composition than the first coating.

Coatings may be provided by various methods, including pre-misting, which may involve: spraying mist into the chamber; capturing the mist in a magnetic field; introducing a stent into the dense, suspended mist; moving the stent around via gentle positioning jets or magnetic field changes; removing the stent after a specified time period; and evacuating the mist from the chamber.

Another coating method may be called coincident coating, and may include: introducing a stent and the mist at the same time; continuously spraying while the stent is suspended in the magnetic field; and turning off the spraying and removing the stent simultaneously.

Another alternative exemplary method may be called pre-loading, and may include: introducing a stent into the chamber; beginning spraying while the stent is suspended in a magnetic field; stopping spraying; and removing the stent from the chamber.

Another alternative exemplary method may be called a continuous coating method and may have the advantage that the solution is reused. This exemplary method may include: spraying mist into the chamber; capturing the mist in a magnetic field; introducing the stent into the dense suspended mist; moving the stent around via gentle positioning jets or magnetic field changes; injecting more solution mist as required to maintain the mist density; and removing the stent after a specified time period. Additionally, a second stent may be introduced into the mist without clearing the chamber and the second stent may be removed after a specified time. This exemplary method may be repeated with additional steps. Furthermore, a density of the solution mist may be monitored with a vision system, which may control the injection of more solution mist as required to maintain the mist density.

Alternative exemplary methods of the present invention include the inclusion of magnetic or non-magnetic and metallic or non-metallic materials such as microspheres in the coating solution. Applying a charge to the solution may assist in preventing the droplets in the mist from joining while in suspension in the magnetic field. Since many materials may be used with the exemplary device and may perform differently, non-magnetic and non-metallic microspheres may be used. The microspheres may influence the nature of the coating mist and the nature of the resulting coating. The microspheres may be specifically sized particles of drug to release a predetermined dose from the coating.

This method may be used for other medical devices besides stents, for instance catheters or any other appropriate devices.

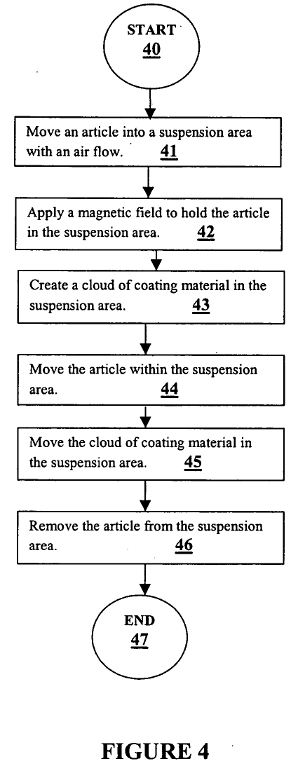

FIG. 4 is a flowchart for performing an exemplary method of the present invention. FIG. 4 begins in start circle 40 and proceeds to action 41, which indicates to move an article into a suspension area with an air flow. From action 41, the flow proceeds to action 42, which indicates to apply a magnetic field to hold the article in the suspension area. From action 42, the flow proceeds to action 43, which indicates to create a cloud of coating material in the suspension area. From action 43, the flow proceeds to action 44, which indicates to move the article within the suspension area. From action 44, the flow proceeds to action 45, which indicates to move the cloud of coating material in the suspension area. From action 45, the flow proceeds to action 46, which indicates to remove the article from the suspension area. From action 46, the flow proceeds to end circle 47.

In alternative exemplary methods, the sequence of steps may be reordered, steps may added or removed, and steps may be modified. For instance, either of actions 44 and 45 may be removed and action 43 may be performed before either or both of actions 41 and 42. Alternatively, action 43 may be performed continuously throughout the method. The method shown in FIG. 4 may be repeated in a continuous fashion, and/or may be performed on more than one article at a time.

As used herein, the term “therapeutic agent” includes one or more “therapeutic agents” or “drugs”. The terms “therapeutic agents”, “active substance” and “drugs” are used interchangeably herein and include pharmaceutically active compounds, nucleic acids with and without carrier vectors such as lipids, compacting agents (such as histones), virus (such as adenovirus, andenoassociated virus, retrovirus, lentivirus and α-virus), polymers, hyaluronic acid, proteins, cells and the like, with or without targeting sequences.

The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, cladribine, 5-fluorouracil, methotrexate, doxorubicin, daunorubicin, cyclosporine, cisplatin, vinblastine, vincristine, epothilones, endostatin, trapidil, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-araginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet inhibitors and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include monocyte chemoattractant proteins (”MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered.

Any of the therapeutic agents may be combined to the extent such combination is biologically compatible.

Any of the above mentioned therapeutic agents may be incorporated into a polymeric coating on the medical device or applied onto a polymeric coating on a medical device. With respect to the type of polymers that may be used in the coating according to the present invention, such polymers may be biodegradable or non-biodegradable. Non-limiting examples of suitable non-biodegradable polymers include polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulosic polymers such as cellulose, cellulose acetate, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), polyorthoesters, maleic anhydride copolymers, and zinc-calcium phosphate.

In a preferred embodiment, the polymer is polyacrylic acid available as HYDROPLUS® (Boston Scientific Corporation, Natick, Mass.), and described in U.S. Pat. No. 5,091,205, the disclosure of which is incorporated by reference herein. In a more preferred embodiment, the polymer is a co-polymer of polylactic acid and polycaprolactone.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The coating can be applied to the medical device by any known method in the art including dipping, spraying, rolling, brushing, electrostatic plating or spinning, vapor deposition, air spraying including atomized spray coating, and spray coating using an ultrasonic nozzle.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters, guide wires, balloons, filters (e.g., vena cava filters), stents, stent grafts, vascular grafts, intraluminal paving systems, implants and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, and the like.

While the present invention has been described in connection with the foregoing representative embodiment, it should be readily apparent to those of ordinary skill in the art that the representative embodiment is exemplary in nature and is not to be construed as limiting the scope of protection for the invention as set forth in the appended claims.

What is claimed is:

1. A non-contact coating system, comprising:
a high-powered magnet adapted to suspend an object; and
an arrangement for creating a coating mist in a suspension zone of the magnet.

2. The system of claim 1, further comprising an air mover adapted to at least one of insert the object into the suspension zone, move the object within the suspension zone, and remove the object from the suspension zone.

3. The system of claim 1, further comprising a magnetic levitation arrangement adapted to at least one of insert the object into the suspension zone, move the object within the suspension zone, and remove the object from the suspension zone.

4. The system of claim 1, further comprising a robotic arm for at least one of placing the object in the suspension zone, dropping the object into the suspension zone, and retrieving the object from the suspension zone.

5. The system of claim 1, further comprising a conveyor system for at least one of dropping the object into the suspension zone and recovering the object after the magnet is powered down.

6. The system of claim 1, further comprising:
a vision system adapted to track at least one of the coating mist and the object; and
a processor electrically coupled to the vision system.

7. The system of claim 1, further comprising a weigh station adapted to weigh the object at least one of before and after the object is suspended in the suspension zone.

8. The system of claim 1, further comprising an arrangement for drying the object, the arrangement for drying the object comprising at least one of an air-flow arrangement, a heater, and a vacuum chamber.

9. The system of claim 1, wherein the arrangement for creating the coating mist comprises at least one of a low pressure nozzle, an ultrasonic nozzle, an electrostatic arrangement for imparting an electrostatic field to a mist, a magnetic levitation mist movement arrangement, and an airflow mist movement arrangement.

10. The system of claim 1, wherein the high-powered magnet is at least one of liquid cooled and donut-shaped.

11. A method for holding at least one device during a coating process, comprising:
applying a magnetic field to the at least one device, the magnetic field causing the at least one device to be suspended in a coating zone; and
contacting a coating material with the at least one device.

12. The method of claim 11, further comprising drying the coating material on the at least one device, the drying achieved by at least one of waiting a predetermined time period, creating a vacuum around the at least one device, heating the at least one device, and flowing a gas over the at least one device.

13. The method of claim 11, wherein the magnetic field is applied by a liquid cooled magnet.

14. The method of claim 11, further comprising moving the at least one device at least one of into, out of, and within the coating zone by at least one of an airflow arrangement and a magnetic levitation movement arrangement.

15. The method of claim 11, wherein the operation of contacting the coating material with the at least one device comprises at least one of spraying the coating material, transporting a mist of the coating material, and aerosolizing the coating material with an ultrasonic nozzle.

16. The method of claim 11, wherein the applying of the magnetic field to the at least one device and the contacting of the coating material with the at least one device are performed simultaneously.

17. A medical appliance having a coating applied by a method, the method comprising:
positioning the medical appliance in a coating zone;
suspending the medical appliance in the coating zone by applying a magnetic field; and
contacting the medical appliance with the coating.

18. The medical appliance of claim 17, wherein the contacting of the medical appliance with the coating further comprises at least one of spraying the coating with a low pressure spray nozzle, aerosolizing the coating with an ultrasonic nozzle, and providing an electrostatic charge to a mist of coating material.

19. An article of manufacture produced by a method, the method comprising:
moving the article into a suspension area;
applying a magnetic field to hold the article in the suspension area; and
creating a cloud of coating material in the suspension area.

20. The article of claim 19, wherein the moving of the article into the suspension area is assisted by at least one of a magnetic levitation arrangement and an air-flow arrangement.

21. The article of claim 19, wherein the method further comprises moving the article out of the suspension area by at least one of a magnetic levitation arrangement and an air-flow arrangement.

22. The article of claim 19, wherein the method further comprises moving the article within the suspension area by at least one of a magnetic levitation arrangement and an air-flow arrangement.

23. The article of claim 19, wherein the method further comprises moving the cloud of coating material in the suspension area by at least one of adjusting the magnetic field, creating a further magnetic field, and creating an air-flow in the suspension area.

24. A method for applying a coating to an object, comprising:
suspending a mist of the coating in a magnetic field; and
dropping an object through the mist.
